(19) United States
(12) Patent Application Publication
(10) Pub. No.: US 2026/0341440 A1
(43) Pub. Date: Oct. 29, 2026

(54) REAL WORLD DATA BASED SUPPORT FOR GENERATING CLINICAL REPORTS

(71) Applicant: Smart Reporting GmbH, Munich (DE)

(72) Inventors: Su Hwan KIM, Munich (DE); Sigrid AUWETER, Puchheim (DE)

(21) Appl. No.: 18/997,336

(22) PCT Filed: Jul. 22, 2022
(86) PCT No.: PCT/EP2022/070625

§ 371 Date: Jan. 21, 2025

(51) Int. Cl. G16H 15/00

(52) U.S. Cl. CPC G16H 15/00

(57) ABSTRACT

A computer-implemented method, a computer system, a computer program product, and a computer-readable storage medium for supporting the creation of a structured medical findings report is introduced. A case-specific parameter set is determined based on the existing data structure elements in such a way that predetermined data structure elements associated with the at least one findings entry of the structured medical findings report are assigned to each of the parameters of the parameter set including their parameter values. Actions are identified which are documented in previous structured medical reports which are stored in a findings report database and are determined therein by means of the parameter set and values and ontology codes assigned to its parameters, as well as the information of an ontology database. An information overview comprising information about the determined actions as well as the parameters of the determined parameter set and values is generated and visualized.

### TECHNOLOGICAL FIELD

The invention relates to a computer-implemented method, a computer system, a computer program product, and a computer-readable storage medium according to the preambles of claims 1, 14, 17, and 18, respectively.

### BACKGROUND

When creating a medical report in a non-supported reporting system, the author is solely dependent on his or her knowledge and experience in the respective medical field. This can be particularly challenging in the case of multimorbid patients. Against this background, systems for computer-assisted decision support (clinical decision support system, CDSS) have been developed. Such systems offer a user, for example, suggestions for possible findings, further courses of action, or relevant background information such as standard operating procedures (SOPs) and lexical knowledge.

The systems usually draw on textbook knowledge as well as published evidence from clinical studies. Such studies usually provide high-quality clinical evidence, but are also associated with some drawbacks for daily practice. For example, the underlying studies are usually expensive and time-consuming. Months or even years can pass between the start of a study and its publication due to the duration of the study, the evaluation and the review process. Further time passes until published study results find their way into knowledge databases or textbooks. A further disadvantage is that the patient population included in a study is not always representative of the population occurring in actual clinical practice.

Against this background, it has been proposed to make direct use of everyday clinical data without subjecting them to prior trials. Such so-called real-world evidence or real world data approaches are intended to complement the results of conventional clinical trials by correlating treatment approaches and patient outcomes in representative patient populations and under real conditions. Such results are available much more quickly and at lower cost, but do not have the quality of clinical trial results. However, the real-world evidence/data approach is a good means of bridging the disadvantages associated with clinical trial results making this approach very well suited for use in the CDSS already mentioned.

U.S. Pat. No. 10,978,196 B2 describes in the context of psychiatric disorders combining molecular data of a patient comprising several nucleic acid sequences with clinical data of the same patient. A report on these data then provides, among other things, a recommendation for medication associated with the nucleic acid sequences, using cohort data and clinical knowledge from a knowledge base (e.g., from guidelines or scientific publications).

US 2019/0198179 A1 describes the prediction of response to a drug based on real-world evidence data. Based on the available real-world evidence data, patient subgroups and a Patient Similarity Network are formed. Using a network localized regression analysis approach, the Patient Similarity Networks and the patient subgroups are then used to predict response to a drug for the patient being treated.

US 2019/0131016 A1 describes the calculation of a risk score using artificial intelligence for whether an observed lung nodule is malignant. For this purpose, in the event of a positive radiological finding of a pulmonary nodule, biomarkers as well as other clinical parameters are determined by means of specimen collection. A composite risk score is then determined from the values of the biomarkers and the patient's clinical parameters. Comparison of the patient-specific risk score with reference data from a patient population is then used to determine whether the pulmonary nodule is malignant or benign.

The article “Integrating Clinical Knowledge and Real-World Evidence for Type 2 Diabetes Treatment” (Xingzhi Sun et al., AMIA Annu Symp Proc. 2019; 2019:838-847; https://www.ncbi.nlm.nih.gov/pmc/articles/PMC7153065/) describes combining clinical knowledge (e.g., from guidelines) with real-world evidence. Based on clinical knowledge, there may be multiple recommended medication options for different case constellations. Based on efficacy and subgroup analyses of real world evidence data, a prioritization of the different medication options is established.

A prerequisite for all these approaches is the existence of a curated database that allows appropriate data mining. Another important and limiting prerequisite for an application in everyday clinical practice is that sufficient time is available. Decisions have to be made quickly, and due to high patient volumes, the time available per patient is often short. In everyday clinical practice, therefore, the best possible integration into the clinical processes is necessary for the most time-efficient procedure possible.

Therefore, in view of better usability, US 2018/0121617 A1 describes a device for providing a user interface for treatment decisions. Within this user interface there are different windows. In a first patient data window, the user enters data on the patient's identity, profile, and diagnosed medical conditions. Based on this data, a patient population (or subpopulation) is searched that matches this patient. Based on the data on these patient populations, information on treatments such as efficiency, adherence or costs is then determined and this information is displayed to the user in a second so-called report window.

### SUMMARY OF THE INVENTION

It is desirable to further improve the construction and querying of databases for real world data as well as their integration into the clinical workflow.

The invention solves this problem with the method according to claim 1, the computer system according to claim 14, the computer program product according to claim 17, and the computer-readable medium according to claim 18.

One aspect of the invention relates to a computer-implemented method for supporting a generation of a structured medical report. Such a structured medical report comprises user-generated medical report entries/findings entries and a machine-readable hierarchical data structure. At least one element of the data structure and an ontology code encoding the findings entry are assigned to each user-generated findings entry by a computer. The method comprises the following steps:
a. on the user side, creating at least one findings entry within a reporting software environment;
b. on the computer side, determining a case-specific parameter set based on the existing data structure elements in such a way that each of the parameters of the parameter set is assigned predetermined data structure elements associated with the at least one findings entry;
c. on the computer side, assigning parameter values stored in the assigned data structure elements to the parameters of the parameter set;
d. on the computer side, identification of actions documented in previous structured medical reports,
   wherein the previous findings reports are stored in a findings report database and are determined within the findings report database by means of the parameter set and the parameter values and ontology codes assigned to its parameters, as well as the information of an ontology database comprising ontology codes and their systematic and factual relationships;
e. on the computer side, generation of an information overview comprising information about the determined actions as well as the parameters of the determined parameter set and the parameter values assigned to them; and
f. on the computer side, visualizing the information overview within the reporting software environment.

Another aspect of the invention relates to a computer system for supporting the generation of a structured medical report as described above. The system thereby comprises:
a findings report database comprising a plurality of structured findings reports comprising the documentation of at least one action;
an ontology database comprising ontology codes and systematic and factual relationships between different ontology codes; and
a processing unit adapted
   for providing a reporting software environment adapted for displaying, creating, and editing structured findings reports; and
   for performing the steps of at least one of methods described herein.

Another aspect of the invention relates to a computer program product comprising machine-readable instructions for executing one of the methods described herein on a computer.

Another aspect of the invention relates to a computer-readable medium comprising computer-readable instructions that, when executed by a computer, cause the computer to perform one of the methods described herein.

In the following, aspects and embodiments of the invention will be discussed. The details and resulting advantages of the invention will appear from the following description. Where appropriate, reference is made to the corresponding drawings, which show preferred embodiments of the invention for illustration purposes. However, these embodiments do not necessarily represent the full scope of the invention.

### BRIEF DESCRIPTION OF THE DRAWINGS

In the figures:

FIG. 1 illustrates a flow diagram of steps of a method according to the invention;

FIG. 2 shows an example of a structured findings report;

FIG. 3 shows an example of an information summary;

FIG. 4 schematically illustrates a system according to the invention.

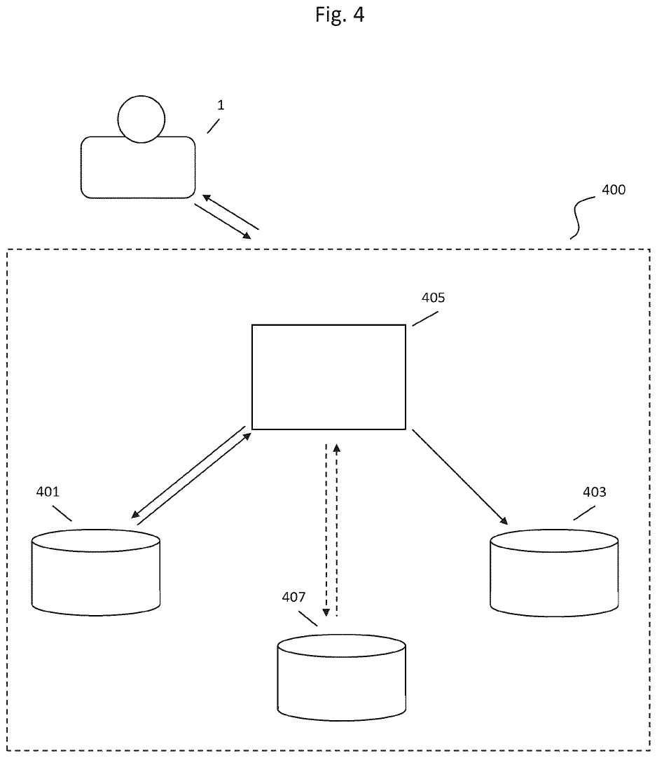

The order of the steps described below can be varied if necessary. In addition, the individual steps can be performed individually or as combinations of a selection of the individual steps.

One aspect of the invention relates to the creation and use of structured medical findings reports. A structured medical report comprises at least entries in the medical report (medical report entries, finding report entries, findings entries) and a data structure comprising data structure elements assignable to the medical report entries. The findings report entries comprise text entered by the user, parameter values entered or selected, images, videos and/or audio recordings. The data structure elements can be assigned to the findings report entries and whatever information is contained in the findings report entries is stored in them. Thus, there is a correspondence between findings report entries and data structure elements. For example, the data structure elements may be the nodes and leaves of a tree structure, arrays or linked lists. In particular, the data structure makes it possible in the context of the invention to rapidly build and, if necessary, expand databases useful for the invention, to determine parameter sets, e.g. for databanks queries, and to efficiently perform database queries.

For the user, primarily the entries of the findings report are visible and editable. However, these have machine-readable equivalents in the elements of the specific data structure i.e. the data structure elements. The data structure thus provides a structure for the findings report and importantly makes its content and meaning machine-readable and analyzable in an effective and automated manner.

Coming back to the findings report, these entries concern different information. For example, a findings report entry may concern a laboratory value such as the leukocyte count or a radiological finding such as the size of the penumbra after an ischemic stroke or general patient information like the patient's date of birth. Different entries can thus concern completely different categories of information. Identifying corresponding entries in different diagnostic reports can be challenging, especially when the structural makeup of the reports is heterogeneous, and thus time consuming and error prone.

In addition, the content of the information contained in a findings report entry can be expressed linguistically differently for the same semantic meaning. For example, “pneumothorax; side: right” semantically means the same as “right-sided pneumothorax.” Thus, there are at least two ways of expressing the same meaning or concept. In particular, heterogeneity of denominations is present when reports are written in different languages (e.g., Turkish and German). A correspondence of semantic meaning is easy to grasp for a technically trained (and possibly linguistically proficient) human reader, but may not be so for a machine system.

With regard to the two points mentioned above, the use of structured findings reports offers advantageous. Due to the structure of the findings reports, which is predefined by the data structure, an unambiguous assignment of findings report entries between reports is possible-even across a large number of reports. Since the findings report entries are clearly assigned to data structure elements, corresponding entries in different findings reports can be identified by identifying identical data structure elements. The same type of information can, thus, be reliably retrieved across different findings reports.

Moreover, by using ontologies, a unique meaning can be assigned to the concrete entries independent of the concrete formulations or language used in each case. An ontology code is only dependent on the respective medical meaning, not on the formulation or language used. This makes structured diagnostic reports well suited for creating, extending and querying databases and especially for data mining.

Another important aspect of using unique database entries is that they make it easy to determine already during the creation of a findings report whether the user has already entered certain information and, thus, this information is available, as well as to read out the content of this information and unambiguously determine its meaning.

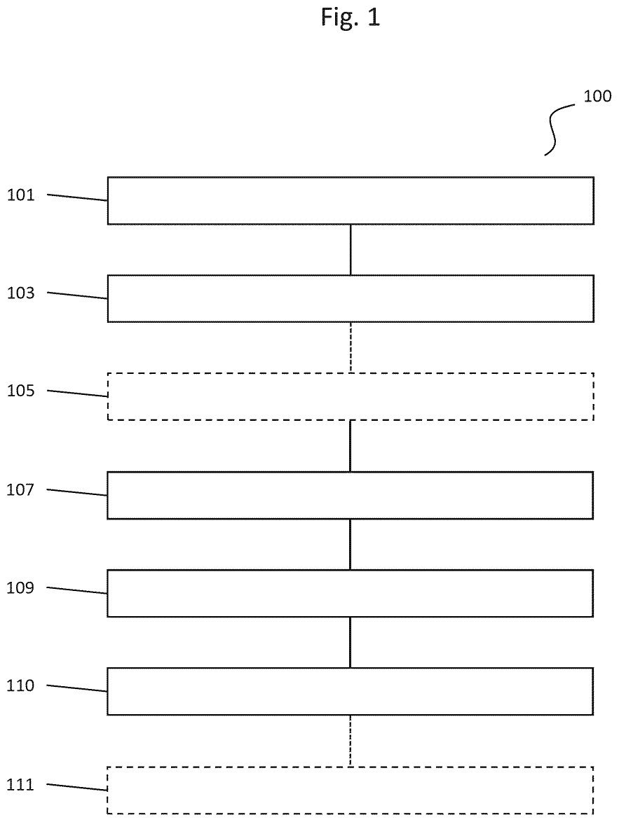

FIG. 1 shows the steps of a method 100 according to a preferred embodiment of the invention. In a first step 101, a user creates one or more findings report entries. This is done in a reporting software environment, i.e., for example, by means of a specific software tool that the physician usually uses to create findings reports. The usual input devices, such as keyboard and mouse/trackpad and/or microphone, can be used. Furthermore, usual output devices, such as screen or loudspeaker, can be used.

In an optional step (not shown), the user can first select a report template from a number of report templates. The structure of the report is then defined by this report template, i.e. specified by it. These templates are created by experts, for example, and therefore represent knowledge models that have been tested by experts and contain all relevant parameters that need to be specified. This ultimately promotes standardization of data entry. Thus, in a preferred embodiment, a library of such templates may be provided, with the individual templates having been created, e.g., by experts, a clinic or, in an optional preceding step, by users themselves.

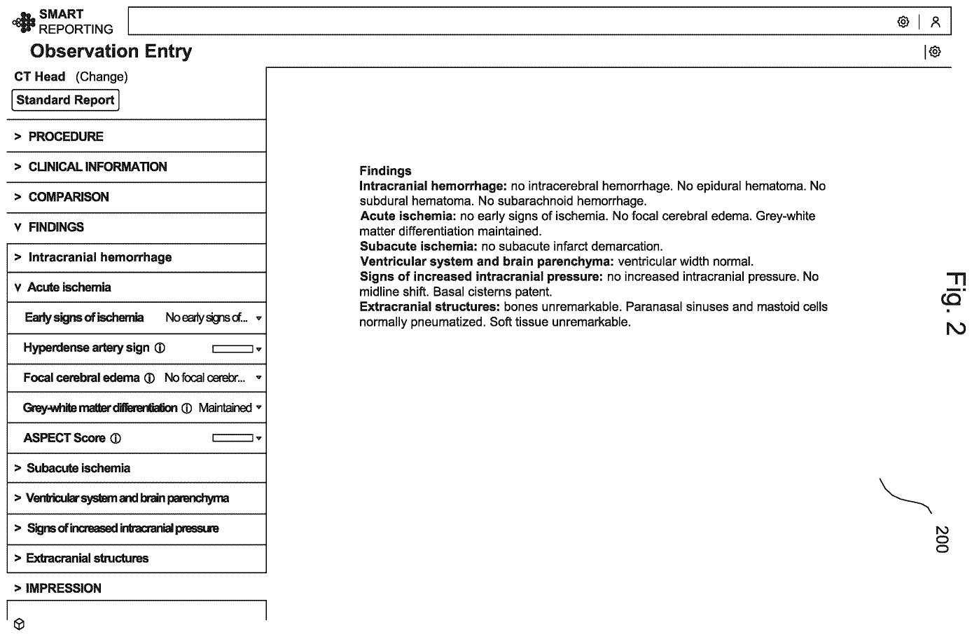

In one embodiment, in any of the two cases, i.e., with or without the use of a template, the structured findings report contains different sections for example, a section for “Findings/Observations,” which contains in particular clinically relevant observations made during an examination, and a section for “Recommendation/Plan,” which contains in particular concluding recommendations or a plan of action for further clinical care. E.g., the latter may contain recommendations or action plans for further diagnostics, if necessary (e.g., follow-up chest CT scan in 6 months), therapy (e.g., increase drug dose to 20 mg, 2×/day), or other (e.g., patient discharge). FIG. 2 shows an example 200 of a structured report of findings.

Back to FIG. 1: on the computer side, a case-specific parameter set is then determined in step 103. A parameter set is a set consisting of one or more parameters. For the sake of clarity and preciseness, the parameters as abstract variables are distinguished herein from the associated corresponding concrete parameter values. For example, the parameters oxygen saturation (sO2) or smoking status formally distinguished from the associated parameter values (e.g., 96% and 2 packs per day). A parameter set then consists of one or more parameters that are assigned to specific data structure elements. This ensures that each of the parameters and, if applicable, its associated parameter value can be read out from at least one findings report entry or the associated data structure element. Thus, there is a unique correspondence between the parameters of the parameter set and the data structure elements of the structured findings report. In addition, the parameter set can also define ranges for each of the parameters within which the assigned parameter values may lie in the sense of a variation range.

A parameter set can be determined based on the findings report template, e.g., the type of the template, and/or the findings entries entered by the user, i.e., patient-specific information entered by the user. For example, a fixed parameter set can be assigned in advance to each specific findings report template, i.e., each type of template corresponds to a specific parameter set. This assigning can be done by experts, a clinic or, in a preceding step, by the user himself. In both cases, the determination of the parameter set is ultimately based on the existence and state or content of data structure elements in the structured findings report.

Alternatively, or additionally, the parameter set may be determined based on a selection of parameters made in previous cases for a database query. In one embodiment, an algorithm is trained to determine the most frequently used configuration parameters or parameter sets for each template. Furthermore, a threshold value for the frequency of use can be specified, above which a parameter is included in the default configuration or parameter set (e.g., more than 50% of users included “age” in the configuration). The selection of a specific findings report template triggers the automatic selection of the predetermined parameter set based on the findings report entries.

Alternatively, or additionally, the parameter set can also be defined and/or modified during the creation of the entries. For example, a rule system can be implemented that determines a parameter set based on an if-then analysis of the entries made by the user and/or adds, changes or removes parameters from a parameter set based on these rules. In both cases, i.e., in the case of parameter sets created in advance or during creation of the report, the determination can be done in two ways. Either, in one embodiment, the data is reviewed by experts in order to determine a system of rules, e.g., a decision tree, base on that the parameter sets will be determined. Or, in another embodiment, the determination is done by means of artificial intelligence (AI). Such an AI can be/has been trained, e.g., on the basis of previous findings reports for which used/fixed parameter sets used in this case are available. For example, in one preferred embodiment, a database stores the automatically suggested parameter set and displays the statistics based on it. Additionally, the database logs/stores if and which parameters have been adjusted or added by the user. Based on the correlations between the parameter set and the decision-making process, the automatically suggested parameter sets can be optimized in such a way that they become better and better and require less and less additional configuration by the user. E.g., if many users always add an additional parameter/filter (“Period: within the last 30 days”), then the AI system learns that this parameter is important and adds it to the parameter set. Finally, some embodiments use a combination of the two approaches for the determination of the parameter sets described above.

In In an optional step 105, the user checks and, if necessary, modifies the default query configuration (default parameter set). This allows the user/physician to query the data of the patient population of interest by manually configuring the relevant parameters, i.e., modifying and/or adding to the systems default. More complex query configurations, e.g., including a temporal aspect, are also possible (e.g., “search out all patients who underwent procedure X in the 30 days prior to the current reporting” or “search out all patients who received an anticoagulant medication in the last 24 hours”). Possible user-configurable parameters in this context include:
patient metadata (hospitalization, age, gender, ethnicity, etc.);
exam metadata (timestamp of exam/report, clinical question/indication of exam; e.g., “search out all patients in whom CT angiography was ordered for the indication “exclusion/confirmation of pulmonary embolism”);
clinical parameters such as parameters of the present findings report (i.e., the one to be prepared) and/or
clinical parameters such as parameters of previous reports (these can be important, because information from previous findings (e.g., previous diseases) could influence which parameters need to be additionally considered for this specific patient; for example, patients with liver cirrhosis typically have poor blood clotting—in these patients, it would be interesting to see whether a particular therapy increases the likelihood of bleeding (outcome)).

In another optional, additional step, the parameter sets used for the database query are recorded and saved in the generated structured findings report and/or in a separate document. In a further embodiment, the aforementioned training of a model for generating the parameter configuration is performed on the basis of the parameter sets used for the database query. For example, the stored parameter sets can be used as training data sets. In particular, as described above, it is possible to implement the automated generation of parameter sets by means of artificial intelligence, e.g., machine learning which has been trained on the basis of findings reports or their entries and the parameter sets that were used for the database query when they were generated.

On the computer side, in step 107, as soon as parameter values are provided by the user for the parameters of the parameter set, the parameter values stored in the data structure elements are assigned to the parameters of the parameter set.

In one embodiment, the step 105 described above and the further optional additional step of storing the parameters occurs after the step 107, and also includes the possibility for the user to manually configure and/or modify their values in addition to the (abstract) parameters.

In a further step 109, actions documented in previous structured medical reports, are identified and in a step 110 an information overview of previous cases and, in particular, actions documented in these cases is created by the computer.

The term actions is to be understood broadly here and also includes suggestions and instructions for actions as well as for findings and/or diagnoses. In particular, such actions include therapeutic measures, such as medications applied or prescribed, and diagnostic measures, e.g., diagnostic steps taken or instructions for further, additional diagnostic steps.

In another embodiment, step 105 and the further optional additional step of storing the parameters occurs after steps 109 and 110 described below, i.e., after identifying documented actions and generating and displaying an information overview. In this embodiment, the user can manually configure and/or modify the parameters and/or their values after inspecting the information overview. Thereupon, based on the modified set of parameters, the identification of actions documented in previous structured medical reports is repeated or updated and a new information overview is generated and displayed to the user, i.e., steps 109 and 110 are repeated. In a preferred embodiment, the user then has the option of modifying the parameter set again, so that steps 105, 107, 109 and 110 can be repeated in a loop-wise manner as often as desired, or until the user makes no further changes to the parameter set or otherwise indicates an end of the loop.

To determine the actions described above, structured findings reports on previous patient cases in which at least one of the actions was recorded are accessed. For this purpose, a database of such structured findings reports is accessed. Depending on the database, these may be patient cases from one or more healthcare facilities at a local, regional, and/or global level. E.g., the database may include reports on patient cases from only a single hospital/healthcare institution or multiple hospitals/healthcare institutions in one or more regions. Examples of how to generate and maintain such a database will be described in detail later.

The database query is performed based on the parameter set and associated parameter values/range of parameter values generated in step 103. In this way, the system can search for reports in which all or at least some of the parameters of the generated parameter set have also been entered and/or in which the associated parameter values match within a specified range. In one embodiment, the search happens findings-centric. This means that all reports that contain the parameters X, Y and Z with the respective values A, B and C will be searched for. In this case it can happen that two or more findings from the same patient are displayed as separate results in the database query.

In another embodiment, the search happens patient-centric. This means that each result of the database query corresponds to one specific patient, even if it includes the results of several cases stored in the database. I.e., the database query happens across all cases that are stored in the database such that all patients for whom there is a finding with parameter X, Y, Z and respective values A, B, C are searched for. The “cases” at the patient level may often be of greater relevance from a clinician's perspective.

The specified range can be defined when the parameter set is created, as described above, or it can be defined by the system during the query. For example, a query in the database may search for all reports where the patients were aged 30-60 years, complained of chest pain and had an oxygen saturation of less than 96%. Thus, in each case, the appropriate options must be selected by the system and/or the user (e.g., “All patients with findings A and B”) and, if necessary, ranges must be defined (e.g., “All patients with a lung tumor 2 to 3 cm in size”). By means of the parameter set, a specific patient population is thus defined, which will be used as a basis for the subsequent query of the findings report database.

This is where the advantage of using a structured database becomes apparent. The fact that the data structure of the findings report to be created corresponds to that of the findings reports stored in the database means that the parameters and their values can be automatically found, read out and compared across several findings reports. Even in the case of differing data structures, if a mapping between the different data structures is available, such a readout and such a comparison is possible. In this case, structured findings reports with different data structures can also be used.

In one embodiment, a database query searches for findings reports that contain all or parts of the parameters of the parameter set. In the case of encoding the entries by ontology codes described above, this query may search for the ontology codes corresponding to the parameters.

In a further embodiment, not only the parameters or their ontology codes are searched for, but only those findings reports are filtered out which not only comprise all or at least parts of the parameters (or ontology codes) of the parameter set, but whose corresponding parameter values additionally match those of the parameter set within a predefined tolerance range. The tolerance range can be determined statically in advance or dynamically during the generation of the findings report entries by the user or the system.

The concrete contents of the findings reports, i.e. the one currently being created and the one stored in the findings report database, are uniquely identified by the ontology codes, for example. It is therefore additionally or alternatively possible to search in the stored findings reports for the ontology codes belonging to the parameter set and to read out and compare the parameter values based on them. The parameters are thus made uniquely identifiable by the ontology codes assigned to them.

In one embodiment, additionally, an ontology database is used when querying the findings report database and generating the information overview. In addition to the pure ontology codes, an ontology database comprises additional information about systematic and factual relationships between the different ontology codes, e.g., in the form of a knowledge model. This allows to search not only for findings reports containing the concrete ontology code(s), but to include more broadly findings reports containing information related to the concrete ontology code in the search. For example, the finding femur fracture has the SNOMED CT ontology code 71620000, while an open femur fracture has the SNOMED CT ontology code 28576007 assigned to it. The SNOMED CT ontology database can now be used to obtain the information that the femur fracture or its code as parent is hierarchically superior to the open femur fracture or its code as child, i.e. the former encompasses the latter. In a database query based purely on the ontology code of the femur fracture, cases of an open femur fracture that actually fall under it would therefore incorrectly not be included in the query. If, on the other hand, information from the corresponding ontology database (SNOMED CT in the above example) is also included in the database query, its subcategories can also be included for a category. For example, a reasoning engine receives input parameters and provides comprehensive results based on logical inferences that can be made using information from the ontology database. The system then recognizes, e.g., [lung tumor] as a subtype of [tumor] and includes all [lung tumors] in the results of [tumor].

Thus, in the above-described case of a query based on individual, specific ontology codes alone only those findings reports in the findings report database are determined (filtered), which contain data structure elements represented by the same specific ontology codes. In contrast, when taking into account the additional information of the ontology database, findings reports can be determined that contain data structure elements encoded with ontology codes that differ from the ones corresponding to the parameters of the parameter set, but are factually and/or systematically related to them.

Regardless of how the database query is performed in detail, in step 110 an information overview is created from the findings reports determined by the database query. Since the database query is based on the aforementioned set of key parameters, the findings reports determined in step 109 represent cases that are comparable to the one currently present. For example, the determined findings reports may report on cases with the same or related medical conditions, comparable age groups, comparable medical histories, comparable life circumstances, or comparable risk factors. Step 109 further includes extracting actions recorded in the determined findings reports are These may be the actions listed above, which are not exhaustive, e.g., suggestions and instructions for action. For example, in imaging studies (e.g., CT) to evaluate a tumor disease (e.g., renal tumor), the imaging findings are described. At the end, often a recommendation for the next steps or actions is given, e.g.
Follow-up examination in 3 months (repeat the same examination)
Biopsy to confirm the diagnosis
PET/CT to complete staging (staging is the complete clarification of the tumor stage, which typically occurs relatively soon after diagnosis-this includes, for example, the search for possible metastases)

The recommended procedures or actions recommended in a findings report depends on the clinical information about the patient, but also on the radiological findings of the current examination.

Based on the extracted actions, in step 110 an information overview of these actions is created.

The created information overview can contain and visualize a variety of different information. For example, the information overview can be implemented in the form of an analysis dashboard. The content of the information overview can include general information and/or statistical information on documented actions and patient outcomes. Statistical information comprises count statistics, mean and median values, ROC-values, correlations, in particular R-values, and errors like standard deviations. However, this list is not exhaustive and the term statistical information includes all parameters, values and quantities commonly known to the skilled person in the field of clinical statistics.

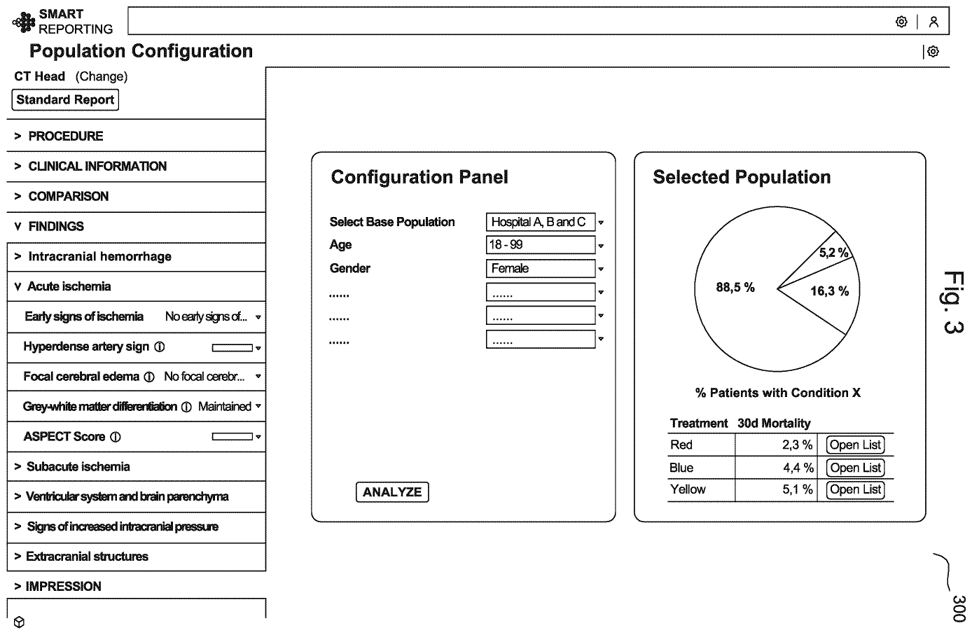

For example, the information overview can include:
Total number of patients included in the analysis.
Proportion of a subpopulation included in the analysis to a total population (example. “Selected subpopulation represents 5% of patients with type II diabetes and 0.2% of all patients documented at Hospital A during the selected time period.”).
Proportions of the selected subpopulation for which available recommendation options were made in each case (reflects recommendations/plans defined by previous physicians in similar cases). FIG. 3 shows an example 300 of such an information summary. Ex. “Treatment [Red] was recommended for 88.5% of the subpopulation, treatment [Blue] was recommended for 5.2%, and treatment [Yellow] was recommended for 16.3%”.
Standardized outcome metrics for each recommended option: e.g., 30-day survival rate (see FIG. 3). The outcome metrics can be obtained, on the one hand, from literature values (e.g., from studies, textbooks, etc.) and/or, as real-world data, from the findings reports of the database itself or from a patient outcomes database. The latter can, for example, be recorded in the findings report itself or in a follow-up report. Another possibility is to specify both literature values and real-world values for the outcome metrics.

The physician now has the option of analyzing the results of the database query via the information overview and, on this basis, deciding which recommendations and/or instructions for action to issue in the report to be generated. In one embodiment, the user is offered a list of available options for further actions for this purpose. This list may include actions resulting from the database query and/or actions specified by the template for the present findings report and/or from knowledge databases (e.g., textbooks, guidelines, etc.). The list can also be dynamically updated during the report generation process by adding or omitting list entries depending on existing parameters and, if applicable, their values (e.g., option A is only displayed if the patient's age is less than 60 years). A criterion for when an action is added to the list or can be considered suggested may, for example, be the relative frequency with which an action was ordered and/or performed in the past. An alternative or additional criterion is the outcome metric. E.g., there can be a rule that only the most frequently given action or the action with the best outcome metric is suggested by the system. However, several actions, e.g., the two, three, four or five most frequently specified actions or the two, three, four or five actions with the best outcome metric can also be suggested by the system. Finally, a weighted or unweighted combination of both criteria can also be used for such a selection. For example: the overall rating is composed of 30% frequency and 70% patient outcome metric.

In a further embodiment, it is also recorded when the user makes a selection other than that suggested by the results of the database query. He may then be prompted to provide a reason for this deviation. This additional information/data may be used to optimize the above list of available recommended options. The answer to this question can be given by the user in a structured way (selection of several possible options) or in an unstructured way (free text). For example, the user indicates that thrombolytic therapy is contraindicated because the patient is taking an anticoagulant medication that increases the risk of bleeding. This entered information (e.g., as free-text data) can then be further analyzed for inclusion in the provided list of available options. As a consequence, in the future, for a patient on anticoagulant medications, “thrombolytic therapy” may be excluded from the list of suggestions. In a preferred embodiment, the reasons for the discrepancy are recorded and stored in the findings report and/or in a separate document. In a further embodiment, a model for generating suggestions for action instructions is trained based on the final given instructions for action and, if applicable, the recorded reasons for a deviation from the instructions for action suggested by the information overview. In particular, the automated generation of suggestions for instructions for action can be implemented by using artificial intelligence, e.g., machine learning, which has been trained on the basis of findings reports and recorded reasons for deviations from the instructions for action suggested by the information overview. Thus, the parameter sets could help to explicitly present the partly subconscious criteria for these recommendations and thus facilitate this decision (especially for more inexperienced physicians).

In a further embodiment, a structured findings report comprising at least one instruction for an action is generated in an additional optional process step 111. The findings report is then added to the findings report database. Since the diagnostic reports in the database have the same data structure as the newly generated diagnostic report or a data structure compatible with it, they can be added to the database without further adjustments or revisions.

Another aspect of the invention relates to a computer system 400 for supporting a generation of a structured medical report.

Such a system 400 includes a findings report database 401, as described above, that includes at least one of the findings reports discussed above comprising a documentation of at least one action, e.g. a suggestion or instruction for an action. Such a database may reside on one or more mass storage devices, a server, or on a network, and in particular may be one or more systems such as a picture archiving and communication system (PACS), a radiology information system (RIS), a hospital information system (HIS), and an electronic medical records (EMR) database, or a combination thereof. In particular, the database may consist of multiple networked databases and archiving systems located at different sites. Thus, herein, the findings report database includes from a single hard drive to a hospital server or the aforementioned systems to a cross-regional networked system consisting of several of the aforementioned storage options.

In addition, the system 400 includes an ontology database 403. The ontology database 403 may include one or more established or user-generated ontology databases. Examples of established ontology databases include RadLex, SNOMED-CT, LOINC, ICDor CPT. Ontologies are often also referred to as classification system, taxonomy, vocabulary or (clinical) terminology. In addition to the mere ontology codes, an ontology database also includes an ontology knowledge model that captures the systematic and factual relationships between the individual codes (see also above). The ontology database therefore not only provides information about the technical meaning of the codes, but also, in the sense of a medical concept, about their classification in a medical system and possible context, such as comorbidities.

Finally, the system 400 comprises a processing unit 405. The processing unit 405 is arranged to provide the diagnostic reporting system for generating the diagnostic reports (not shown). Thus, it is arranged to execute corresponding program instructions for providing a software environment for visualizing, processing and creating structured findings reports. In particular, the processing unit is arranged to create, edit and manage the data structure associated with the structured findings reports. Finally, the processor unit 405 is arranged to generate outputs to the user 1, who is not part of the system but interacts with it, and to receive and process user-side inputs.

The processing unit 405 can, e.g., consist of a single-or multicore processor or multiple physical processors (also referred to as multiple sockets).

The processor unit 405 provides information to the user, e.g., visually by means of a screen or other medium for visual output (not shown) or acoustically, e.g., via loudspeakers (not shown). To the user, the findings report software environment displays, in addition to the findings report to be generated and, if applicable, medical images relevant thereto, in particular the information overview already discussed, i.e., e.g., findings or findings reports from the findings report database, instructions for action and/or patient outcome as well as statistical, if applicable, information thereon.

The user, in turn, enters information into the reporting software environment and thus transmits the information to the system 400 or its processing unit 405 using existing input devices, e.g., keyboard and mouse, microphone, and/or a touch input unit. E.g., the user creates findings report entries, in particular by entering findings. Parameters and recommended actions are entered. In addition, the user can optionally configure or modify the parameter sets and, if applicable, their values and/or value ranges for querying the findings report database.

The processing unit 405 is also arranged to perform the method steps already described. For this purpose, the processing unit 405 exchanges data with the findings report database 401 and the ontology database 403. Thus, the processor unit 405 is arranged to access and read the diagnostic reports stored in the diagnostic report database 401 as part of the database query.

In one embodiment, the processing unit 405 has access to the ontology database 403. The ontology database 403 can exchange (i.e., receive and send) data such as ontology codes with the processing unit 405, as well as transmit information contained in the ontology knowledge model to the processing unit 405 and receive information relevant to the ontology knowledge model from the processing unit 405.

For example, the query might search for findings reports comprising one or more of the data structure elements that are identical to the ones corresponding to the parameters of the parameter set (e.g., via the ontology codes). Also, using the information regarding the structure of the ontology codes comprised in the ontology database, the processing unit 405 can determine ontology codes that are factually and explicit or systematically related to an ontology code corresponding to a parameter of the parameter set. In the query, findings reports comprising also these related ontology codes can be searched for. E.g., if the ontology database has a hierarchical structure not only the ontology code of a parent concept but also the ontology codes of the corresponding children concepts can be searched for.

In one embodiment the processor unit 405 is further arranged to continuously add findings reports created in the reporting software environment to the findings report database 401.

In one embodiment, the processing unit 405 is configured to provide information, e.g., statistical information, regarding the documented actions, e.g., the issued action instructions, contained in the findings reports based on the set of parameters. For example, the processor unit 405 may be arranged to generate count statistics regarding the occurrence of certain action instructions and display them to the user as part of the information overview as described above. In a further embodiment, the processing unit 405 may apply weightings to the found finding reports/actions, such that some finding reports and/or actions contribute more or less to the statistics than others.

Optionally, the system may also include a patient-outcome database 407. In one embodiment, the patient outcome database 407 is part of the findings report database 401. In this case, information about the respective outcomes is either recorded in the stored findings reports themselves and the corresponding information is accessible through them, or the outcome information is separate from the findings reports, e.g., in a separate archive for patient records (electronic health records EHR), but is stored in a database that is part of the findings report database. Thus, the outcome database can be encompassed by the findings report database and vice versa.

In another embodiment, the system 400 includes a patient-outcome database 407 that is separate from the findings report database 401. Here, the processor unit 405 is configured to access information from the patient-outcome database 407 in addition to the information from the findings-report database 401, to attribute this information to the recommended actions derived from the query of the findings-report database 401 and/or to the generated parameter set, and to add the patient-outcome information to the information overview.

In one embodiment, the processor unit is set up to offer the user an interface via which he can modify the parameter set generated by the system and, if necessary, the parameter values and/or parameter value ranges, and to base the database query on the modified parameter set. In one embodiment, the processor is set up to repeat for the user the option of modifying the parameter set and displaying the information overview in a loop until the user makes no further adjustments to the parameter set. This way, the user can adjust the database query as long as she/he deems it necessary. In a further embodiment, the processor unit is further configured to record and store the parameter sets used for the database query in the generated structured findings report and/or in a separate document or file.

In one embodiment, the processor unit 405 is further configured to train a model for generating the parameter configuration based on parameter sets used for previous database queries. In particular, it is thus possible to implement the machine generation of parameter sets by means of artificial intelligence, e.g., a neural network, which has been trained on the basis of findings reports and the parameter sets, value and value ranges used for the database query when these reports were generated.

In one embodiment, the processor unit 405 is adapted to detect when an action entered by the user in the findings report deviates from the one or ones that are suggested by the database query or the information overview, e.g., because they are the most frequently documented or issued or those with the best outcome metrics according to the information overview. In a preferred embodiment, the processing unit 405 then prompts the user to provide reasons for the deviation from the actions suggested by the information overview. These are then recorded and stored in the findings report and/or a separate document.

In one embodiment, the processor unit is also set up to train a model for generating suggestions for actions on the basis of the actions that were ultimately documented and, if applicable, the recorded reasons for a deviation from the actions suggested by the information overview. In particular, it is thus possible to implement the automatic generation of suggestions for actions by means of artificial intelligence, e.g., machine learning, which has been trained on the basis of reports on findings and recorded reasons for deviations from the actions suggested by the information overview.

One aspect of the invention relates to a computer program product. Such a computer program implements, in a suitable programming language, the various embodiments of the methods described above. These instructions may be platform dependent or platform independent. Thus, the computer program product comprises both the source code and any programs obtained by compiling the source code. In particular, the computer program product may be designed to be compiled and/or executed on any of the embodiments of the computer system described above.

Another aspect of the invention relates to a computer-readable medium comprising instructions which, when executed by a computer, cause the computer to perform one of the embodiments of the method described above. The computer-readable medium may be volatile or non-volatile. In particular, it includes magnetic hard disks, solid state hard disks, flash memory (e.g., USB sticks) and optical media such as CD-ROM, DVD and/or BlueRay.

1. A computer-implemented method for supporting the creation of a structured medical findings report comprising user-generated findings entries and a machine-readable hierarchical data structure, wherein at least one data structure element and a first ontology code encoding the findings entry are assigned to each findings entry by the computer, the computer-implemented method including steps comprising:
a. on a user side, creating at least one findings entry within a reporting software environment;
b. on a computer side, determining a case-specific parameter set based on existing data structure elements in such a way that predetermined data structure elements associated with the at least one findings entry are assigned to each parameter of the case-specific parameter set;
c. on the computer side, assigning to the parameters of the case-specific parameter set parameter values stored in the assigned predetermined data structure elements;
d. on the computer side, identifying actions documented in previous structured medical finding reports, wherein the previous structured medical findings reports are stored in a findings report database and are determined within the findings report database using the case-specific parameter set and the parameter values and first ontology codes assigned to the at least one findings entry, as well as the information of an ontology database comprising the first and further ontology codes and systematic and factual relationships of the first and further ontology codes;
e. on the computer side, generating an information overview comprising information about the actions as well as the parameters of the case-specific parameter set and the parameter values assigned to the parameters of the case-specific parameter set; and
f. on the computer side, visualizing the information overview within the reporting software environment.

2. The method according to claim 1, comprising:
on the user side, modifying the case-specific parameter set and/or the parameter values assigned to the parameters of the case-specific parameter set to create a modified case-specific parameter set, and
on the computer side, repeating steps d)-e) using the modified case-specific parameter set and the parameter values assigned to the modified case-specific parameter set.

3. The method according to claim 1, comprising:
generating a structured medical findings report comprising documentation of at least one action, and
adding the structured medical findings report to the findings report database.

4. The method according to claim 1, comprising:
generating, on the computer side, statistical information on individual identified actions documented in the findings report database; and
adding the statistical information to the information overview.

5. The method according to claim 4, wherein the statistical information on the individual identified actions comprises at least one count statistic of the occurrence of individual identified actions in the findings report database.

6. The method according to claim 1, in which the identified actions comprise suggestions and/or instructions for actions and/or findings and, in particular therapy suggestions, suggestions for further diagnostic measures and/or suggestions for further findings.

7. The method according to claim 1, comprising:
on the computer side, based on data contained in at least one patient-outcome database, generating information about clinical outcomes attributable to the identified actions; and
adding the information about clinical outcomes attributable to the identified actions to the information overview.

8. The method according to claim 7, wherein the information about clinical outcomes comprises statistical information.

9. The method according to claim 4, comprising determining that a user-documented action in the findings report deviates from being a most frequently documented action and/or an action having a greatest likelihood of achieving a desired clinical outcome:
receiving a decision rationale for the user-documented action deviating from being the most frequently documented action and/or the action having a greatest likelihood of achieving a desired clinical outcome; and
saving the decision rationale.

10. The method according to claim 3, in which a model for generating suggestions for actions is trained using the findings report database.

11. The method according to claim 9, in which a model for generating suggestions for actions is trained using the findings report database and the decision rationale.

12. The method according to claim 1, in which the case-specific parameter set used in the creation of the findings report is stored.

13. The method according to claim 12, in which a model for generating parameter sets is trained on the basis of case-specific parameter sets stored as part of creation of the previous findings reports.

14. A computer system for supporting the creation of a structured medical findings report comprising user-generated findings entries and a machine-readable hierarchical data structure, wherein at least one data structure element and a first ontology code encoding the findings entry are assigned to each findings entry by the computer, the computer system comprising:
at least one memory having stored thereon:
a findings report database comprising a plurality of structured findings reports comprising the documentation of at least one action;
an ontology database comprising the first ontology code and further ontology codes and systematic and factual relationships between different comprised ontology codes; and
a processing unit configured to access the at least one memory and carry out steps comprising:
a. on a user side, creating at least one findings entry within a reporting software environment;
b. on a computer side, determining a case-specific parameter set based on existing data structure elements in such a way that predetermined data structure elements associated with the at least one findings entry are assigned to each parameter of the case-specific parameter set;
c. on the computer side, assigning to the parameters of the case-specific parameter set parameter values stored in the assigned predetermined data structure elements;
d. on the computer side, identifying actions documented in previous structured medical finding reports, wherein the previous structured medical findings reports are stored in a findings report database and are determined within the findings report database using the case-specific parameter set and the parameter values and first ontology codes assigned to the at least one findings entry, as well as the information of an ontology database comprising the first and further ontology codes and systematic and factual relationships of the first and further ontology codes;
e. on the computer side, generating an information overview comprising information about the actions as well as the parameters of the case-specific parameter set and the parameter values assigned to the parameters of the case-specific parameter set; and
f. on the computer side, visualizing the information overview within the reporting software environment.

15. The system according to claim 14, wherein the at least one memory further has
at least one patient-outcome database stored thereon, and wherein the processor is configured to generate and add to the information overview statistical information on clinical outcomes for the individual identified actions based on data contained in the at least one patient-outcome database.

16. The system according to claim 14, wherein the findings report database comprises the at least one patient-outcome database.

17. (canceled)

18. (canceled)
